(19) United States
(12) Patent Application Publication
(10) Pub. No.: US 2025/0170040 A1
(43) Pub. Date: May 29, 2025

(54) PERSONAL CARE PRODUCT COMPOSITIONS COMPRISING A FATTY AMIDE AND METHODS FOR MAKING SAME

(71) Applicant: Ash Ingredients Inc., Glen Rock, NJ (US)

(72) Inventors: Konstantinos M. Lahanas, West Milford, NJ (US); Steven L. Fantano, Hackettstown, NJ (US)

(73) Assignee: Ash Ingredients Inc., Glen Rock, NJ (US)

(21) Appl. No.: 18/961,769

(22) Filed: Nov. 27, 2024

Related U.S. Application Data
(60) Provisional application No. 63/603,910, filed on Nov. 29, 2023.

(51) Int. Cl. A61K 8/42; A61Q 19/00

(52) U.S. Cl. CPC A61K 8/42; A61Q 19/00; A61K 2800/10; A61K 2800/30; A61K 2800/34

(57) ABSTRACT

The present disclosure generally relates to personal care product compositions comprising a fatty amide such as erucamide and/or derivatives thereof having a chemical formula ranging from C₁₈H₄₃NO to C₄₀H₇₉NO for use on hair and/or skin of a user. The disclosure also relates to methods for making such compositions. Such personal care products provide improved biodegradable properties, softer and smoother skin, softer, smoother and shinier hair, and hair with improved body and manageability, while reducing skin irritation and product build-up in the hair and on the skin than personal care products comprising cationic polymers and/or silicone compounds. The formulation of the personal care product may also include one or more additional ingredients such as water, glycerin, hydroxypropylmethylcellulose, glyceryl monostearate, cetyl alcohol, and glyceryl/polyglyceryl-3 caprylic/capric/lactic/lauric acid esters in various concentrations and/or combinations.

### CROSS-REFERENCE TO RELATED APPLICATIONS

This application claims priority to U.S. Provisional Patent Application No. 63/603,910, filed on Nov. 29, 2023, which is incorporated herein by reference in its entirety.

### FIELD

Aspects of the present disclosure generally relate to personal care product compositions comprising a fatty amide such as erucamide and/or derivatives thereof having a chemical formula ranging from C₁₈H₄₃NO to C₄₀H₇₉NO, and methods for making such compositions.

### BACKGROUND

Traditionally personal care products on the market, such as hair conditioners, shampoos, shaving creams, moisturizing lotions and the like often contain cationic polymers or silicone compounds. Cationic polymers, such as quats, and/or silicone compounds have traditionally been used in personal care products as a slip additive to reduce the coefficient of friction and/or static electricity in the user's hair or on the user's skin when the personal care product is used. However, because such compounds are not biodegradable and have a large carbon footprint in their manufacture, their use in personal care products is harmful to the environment. Many of these compounds ultimately end up in the ground or the water supply, for example because they are washed down the drain during showering or bathing, or they are stripped from hair and/or skin during swimming in lakes, rivers and oceans. Disposal of personal care products containing these compounds in landfills also contributes to this problem.

In addition, personal care products using cationic polymers and/or silicone compounds can have undesirable effects on the human body. For example, the use of silicone in personal care products can lead to skin and scalp irritation. Further, cationic polymers and silicone compounds used in hair conditioners and shampoos can lead to residue buildup, leaving a user's hair heavy and limp, in turn making it difficult to manage and style. This may cause a user to shower more frequently, thereby increasing the deposit rate of these compounds into the environment, further damaging the user's hair, and wasting water.

There is a need for personal care products comprising compositions that utilize cationic polymers and silicone compounds to the least extent possible. There is also a need for these products to be formulated to improve biodegradable qualities while maintaining (or even improving) the performance characteristics of the products, such as providing smoother skin; shinier, softer and more manageable hair; less irritation and less product build-up in the hair and on the skin.

### SUMMARY

The present disclosure attempts to solve one or more of the aforementioned needs by providing, in one aspect, personal care product compositions comprising a fatty amide, such as erucamide and/or derivatives thereof having a chemical formula ranging from C₁₈H₄₃NO to C₄₀H₇₉NO. Such erucamide derivatives may include stearyl erucamide. The present disclosure also discloses methods for making such compositions. Personal care products contemplated herein may include, but are not limited to, hair conditioning lotions, hair conditioners, conditioning shampoos, 2-in-1 conditioning shampoos, hair color treating shampoos and/or conditioners, hair color treating 2-in-1 conditioning shampoos, quat-free conditioners, non-foaming shave lotions and shave creams, foaming shave lotions and shave creams, brushless shave creams and lotions, moisturizing lotions and creams, barrier repair lotions and creams, conditioning hand lotions and creams, cleansing wipes, hair detangling sprays, leave-in conditioners, deodorants, deodorant sticks, antiperspirants, antiperspirant sticks, press powder eye shadow, and pigmented products such as make-up, color cosmetics, powders, lipsticks, eye shadows, blush, foundation, etc. that include a fatty amide such as erucamide or its derivatives having a chemical formula ranging from C₁₈H₄₃NO to C₄₀H₇₉NO. The contemplated personal care product formulations may also include one or more other components including but not limited to water, a triol (e.g., glycerin), a surfactant (e.g., sodium lauroyl methyl isethionate), a cleansing agent (e.g., sodium lauroyl sarcosinate), a thickener (e.g., hydroxypropylmethylcellulose), emulsifiers (e.g., glyceryl monostearate), preservatives (e.g. glyceryl/polyglyceryl-3 caprylic/capric/lactic/lauric acid esters), a fatty alcohol (e.g., cetyl alcohol), a fatty acid (e.g., stearic acid), and citric acid, in various concentrations and/or combinations thereof. Other components in the personal care product formulations of the present disclosure may also include a nonionic polymer (e.g., cetyl hydroxyethylcellulose), a fragrance (e.g., lavender, vanilla, etc.), an emollient (e.g., caprylic/capric triglyceride), baking soda (e.g., sodium bicarbonate), magnesium hydroxide, coconut oil, arrowroot powder, shea butter, sunflower wax, in various concentrations and/or combinations thereof.

It is further contemplated that personal care product compositions comprising a fatty amide, such as erucamide or its derivatives having a chemical formula ranging from C₁₈H₄₃NO to C₄₀H₇₉NO may provide benefits to a user, such as smoother skin; shinier, softer and more manageable hair; less irritation and less product build-up in the hair and on the skin than compositions having silicone compounds or cationic polymers. It is therefore contemplated by the present disclosure that a fatty amide is to be used in personal care products in place of or as an alternative to cationic polymers and/or silicone compounds.

The above summary has outlined, rather broadly, some advantages of the present disclosure so that the detailed description that follows may be better understood. Additional features and advantages of the disclosure will be described below. It should be appreciated by those skilled in the art that this disclosure may be readily utilized as a basis for modifying or designing other compositions and formulations for carrying out the same or similar purposes of the present disclosure. It should also be realized by those skilled in the art that such equivalent compositions and formulations do not depart from the teachings of the disclosure as set forth herein and in the appended claims. The novel features, which are believed to be characteristic of the disclosure, both as to its composition and method of making the same, together with further features and advantages, will be better understood from the following description.

### DETAILED DESCRIPTION

The detailed description set forth below is intended as a description of various compositions and methods for making the same and is not intended to represent the only way the concepts described herein may be practiced. The detailed description includes specific details for the purpose of providing a thorough understanding of the various concepts. It will be apparent, however, to those skilled in the art that these concepts may be practiced without these specific details. In some instances, well-known elements are only briefly mentioned or described in order to avoid obscuring the material aspects of the present disclosure.

Reference may be made herein to other United States patents, foreign patents, and/or other technical references. Any reference made herein to other documents is an express incorporation by reference of the document so referenced in its entirety.

The terms “a,” “an,” and “the” as used herein are defined to mean “one or more” and include the singular and the plural unless the context is inappropriate.

As described herein, the use of the term “and/or” is intended to represent an “inclusive OR,” and the use of the term “or” is intended to represent an “exclusive OR.”

The term “comprising” as used herein which is synonymous with “including,” “containing,” or “characterized by,” is inclusive or open-ended and does not exclude additional, un-recited elements of a composition or method steps. The term “consisting of” excludes any element, step, or ingredient that is not otherwise specified. The term “consisting essentially of” limits the scope of a composition or method to the specified materials or steps and those that do not materially affect the basic and novel characteristic(s) of the specified composition or method.

“Derivative” as used herein means any fatty amide having a chemical formula ranging from C₁₈H₄₃NO to C₄₀H₇₉NO, but not C₂₂H₄₃NO.

“Erucamide” as used herein is a fatty amide having a chemical formula of C₂₂H₄₃NO. In one embodiment, the Erucamide is or may be referred to as Cis-13-docosenamide.

“Hair Conditioner” refers to a hair care composition used to improve the feel, texture, appearance and manageability of hair.

“Shampoo” refers to a composition that is applied to the hair of a user, whereby scrubbing and/or otherwise dispersing the shampoo throughout the user's hair in the presence of added water disinfects, deodorizes, cleanses, conditions, and/or moisturizes the hair.

“Lotion” refers to a thick, smooth liquid or semi-liquid preparation designed to be applied to the skin or hair for cosmetic or external medicinal use.

“Cream” refers to a thick, viscous liquid or semi-liquid preparation design to be applied to the skin or hair for cosmetic or external medicinal use.

“Wipes” refers to any pre-treated article used to cleanse the skin or hair of a user.

“Detangling spray” refers to a type of leave-in conditioner that smooths and moisturizes the hair of a user.

“Cleansing” refers to the ability of a personal care product to remove dirt, oil, bacteria, and other extraneous or foreign matter from the skin or hair of a user.

“Conditioning” refers to the ability of a personal care product to provide a soft and smooth feel to the skin or hair, and provide, in the case of hair products, hair that is shiny, has body and is manageable and able to be styled.

“Moisturizing” refers to the ability of a personal care product to hydrate the skin or hair of a user or otherwise minimize a drying effect on the skin or hair.

“Shaving” refers to severing hair from any part of the body close to the skin, including from one's face, head, neck, back, and legs.

“Deodorant” refers to a composition that is applied to the skin of a user, whereby applying the deodorant to the user's skin removes or conceals unpleasant odors from the skin and/or body.

“Antiperspirant” refers to a composition that is applied to the skin of a user, whereby applying the antiperspirant to the user's skin prevents or reduces perspiration.

“Pigmented products” refer to compositions or products that contain pigments, which are colored particles that change or modify the appearance of a material and in this case refer generally to pigmented or color cosmetics (e.g., make-up, powder, lipstick, blush, foundation, eye shadow, etc.). Applying a “pigmented product” to a user's skin or body part causes the skin or body part to change to the color of the pigmented product.

All percentages and ratios are calculated based on the weight of the total composition unless otherwise indicated.

The present disclosure is generally related to personal care products that include erucamide and/or its derivatives which are used on the body, head and/or hair of a user. Formulation of these contemplated personal care products may also include one or more other components such as water, a triol (e.g., glycerin), a surfactant (e.g., sodium lauroyl methyl isethionate), a cleansing agent (e.g., sodium lauroyl sarcosinate), a thickener (e.g., hydroxypropylmethylcellulose), emulsifiers (e.g., glyceryl monostearate), preservatives (e.g. glyceryl/polyglyceryl-3 caprylic/capric/lactic/lauric acid esters), a fatty alcohol (e.g., cetyl alcohol), a fatty acid (e.g., stearic acid), and citric acid, in various concentrations and/or combinations thereof and any other ingredient disclosed herein, as further described below.

Erucamide and its derivatives are primary fatty amides resulting from the formal condensation of the carboxy group of erucic acid with ammonia. Erucamide and its derivatives are commonly used as slip additives in the plastic manufacturing industry, and specifically as a release agent for polyolefins used in rotational and injection molding of items such as syringes, furniture compounds, and automobile compounds. Their use improves mold release, thereby reducing and/or eliminating the use of mold release spray; allows the use of lower clamp and ejection pin pressure; lowers rejection rates caused by deformation and pin marks; improves melt flow characteristics and reduces cycle time; and improves the final “feel” of the molded article and scratch resistance.

The present disclosure details how to use erucamide and its derivatives, which were traditionally used as a slip additive in the plastic manufacturing industry, in personal care products to achieve smoother and less irritated skin, reduced product build-up in the hair and on the skin; and smoother, softer, shinier and more manageable hair.

According to one aspect of the present disclosure, formulations for personal care products containing erucamide or its derivatives may comprise anywhere between about 0.0001% to about 20% by weight of erucamide or its derivatives, based on the total weight of the formulation. In one aspect of the disclosure, the concentration of erucamide or its derivatives may be about 2% of the formulation.

In some embodiments of the present disclosure, formulations for personal care products containing erucamide or its derivatives may comprise water, such as deionized water. Formulations may comprise anywhere between about .0001% to about 98% by weight of water, based on the total weight of the formulation. In one aspect of the disclosure, the concentration of water may be about 87% of the formulation.

In some embodiments of the present disclosure, formulations for personal care products containing erucamide or its derivatives may comprise a triol such as glycerin. Formulations may comprise anywhere between about 0.0001% to about 20% by weight of glycerin, based on the total weight of the formulation. In one aspect of the disclosure, the concentration of glycerin may be about 3% of the formulation.

In some embodiments of the present disclosure, formulations for personal care products containing erucamide or its derivatives may comprise a thickener such as hydroxypropylmethylcellulose. Formulations may comprise anywhere between about 0.0001% to about 20% by weight of hydroxypropylmethylcellulose, based on the total weight of the formulation. In one aspect of the disclosure, the concentration of hydroxypropylmethylcellulose may be about 1% of the formulation.

In some embodiments of the present disclosure, formulations for personal care products containing erucamide or its derivatives may comprise an emulsifier such as glyceryl monostearate. Formulations may comprise anywhere between about 0.0001% to about 20% by weight of glyceryl monostearate, based on the total weight of the formulation. In one aspect of the disclosure, the concentration of glyceryl monostearate may be about 2% of the formulation.

In some embodiments of the present disclosure, formulations for personal care products containing erucamide or its derivatives may comprise a fatty alcohol such as cetyl alcohol. Formulations may comprise anywhere between about 0.0001% to about 20% by weight of cetyl alcohol, based on the total weight of the formulation. In one aspect of the disclosure, the concentration of cetyl alcohol may be about 3% of the formulation.

In some embodiments of the present disclosure, formulations for personal care products containing erucamide or its derivatives may comprise another emulsifier such as glyceryl/polyglyceryl-3 caprylic/capric/lactic/lauric acid esters. Formulations may comprise anywhere between about 0.0001% to about 20% by weight of glyceryl/polyglyceryl-3 caprylic/capric/lactic/lauric acid esters, based on the total weight of the formulation. In one aspect of the disclosure, the concentration of glyceryl/polyglyceryl-3 caprylic/capric/lactic/lauric acid esters may be about 2% of the formulation.

In some embodiments of the present disclosure, erucamide may be used in personal care products in place of or as an alternative to cationic polymers and/or silicone compounds. Additionally, erucamide may be used as a natural and vegetal alternative to silicone elastomer dispersions. Further, other natural and/or vegetal based ingredients may be used as an alternative to silicon elastomer dispersions.

In one embodiment, a hair conditioner formulation according to the present disclosure comprises erucamide or derivatives thereof. The hair conditioner formulation may further comprise one or more of the following ingredients in the weight percentage ranges identified herein: water, glycerin, hydroxypropylmethylcellulose, glyceryl monostearate, cetyl alcohol, and glyceryl/polyglyceryl-3 caprylic/capric/lactic/lauric acid esters in various combinations and concentrations. Specifically, the hair conditioner formulation may comprise one or more of the following ingredients: about 87% of water, about 3% of glycerin, about 1% of hydroxypropylmethylcellulose, about 2% of glyceryl monostearate, about 2% of erucamide or its derivatives, about 3% of cetyl alcohol, and about 2% of glyceryl/polyglyceryl-3 caprylic/capric/lactic/lauric acid esters.

In another embodiment, a 2-in-1 conditioning shampoo formulation according to the present disclosure comprises erucamide or derivatives thereof. The 2-in-1 conditioning shampoo formulation may further comprise one or more of the following ingredients in the weight percentage ranges identified herein: water, hydroxyethylcelluose, sodium C14-16 olefin sulfonate, sodium lauroyl methyl isethionate, sodium lauroyl sarcosinate, cocamidopropyl betaine, glyceryl/polyglyceryl-3 caprylic/capric/lactic/lauric acid esters, polyglyceryl-10 caprylate/caprate, and citric acid. Specifically, the 2-in-1 conditioning shampoo formulation may comprise one or more of the following ingredients: about 40.80% of water, about 1% of hydroxyethylcelluose, about 20% of sodium C14-16 olefin sulfonate, about 10% of sodium lauroyl methyl isethionate, about 10% of sodium lauroyl sarcosinate, about 8% of cocamidopropyl betaine, about 2% of glyceryl/polyglyceryl-3 caprylic/capric/lactic/lauric acid esters, about 4% of polyglyceryl-10 caprylate/caprate, about 0.20% of citric acid, and about 4% of erucamide or derivatives thereof.

In another embodiment, a shave cream formulation according to the present disclosure comprises erucamide or derivatives thereof. The shave cream formulation may further comprise one or more of the following ingredients in the weight percentage ranges identified herein: water, stearic acid, glyceryl/polyglyceryl-3 caprylic/capric/lactic/lauric acid esters, polyglyceryl-10 caprylate/caprate, glyceryl monostearate, cetyl alcohol, and potassium hydroxide. Specifically, the shave cream formulation may comprise one or more of the following ingredients: about 79% of water, about 10% of stearic acid, about 2% of glyceryl/polyglyceryl-3 caprylic/capric/lactic/lauric acid esters, about 2% of polyglyceryl-10 caprylate/caprate, about 2% of glyceryl monostearate, about 1% of cetyl alcohol, about 2% of potassium hydroxide, and about 2% erucamide or derivatives thereof.

In another embodiment, a brushless shave cream formulation according to the present disclosure comprises erucamide or derivatives thereof. The brushless shave cream formulation may further comprise one or more of the following ingredients in the weight percentage ranges identified herein: water, glyceryl/polyglyceryl-3 caprylic/capric/lactic/lauric acid esters, polyglyceryl-10 caprylate/caprate, glyceryl monostearate, and cetyl alcohol. Specifically, the brushless shave cream formulation may comprise one or more of the following ingredients: about 91% of water, about 2% of glyceryl/polyglyceryl-3 caprylic/capric/lactic/lauric acid esters, about 2% of polyglyceryl-10 caprylate/caprate, about 2% of glyceryl monostearate, about 1% of cetyl alcohol, and about 2% of erucamide or derivatives thereof.

In another embodiment, a cleansing wipes formulation according to the present disclosure comprises erucamide or derivatives thereof. The cleansing wipes formulation may further comprise one or more of the following ingredients in the weight percentage ranges identified herein: water, glyceryl/polyglyceryl-3 caprylic/capric/lactic/lauric acid esters, polyglyceryl-10 caprylate/caprate, and sodium stearoyl lactylate. Specifically, the cleansing wipes formulation may comprise one or more of the following ingredients: about 90% of water, about 2% of glyceryl/polyglyceryl-3 caprylic/capric/lactic/lauric acid esters, about 4% of polyglyceryl-10 caprylate/caprate, about 2% of sodium stearoyl lactylate, and about 2% of erucamide or derivatives thereof.

In another embodiment, a hair detangling spray and/or leave-in conditioner formulation according to the present disclosure comprises erucamide or derivatives thereof. The hair detangling spray and/or leave-in conditioner formulation may further comprise one or more of the following ingredients in the weight percentage ranges identified herein: water, propanediol, glyceryl/polyglyceryl-3 caprylic/capric/lactic/lauric acid esters, polyglyceryl-10 caprylate/caprate, and glyceryl monostearate. Specifically, the hair detangling spray and/or leave-in conditioner formulation may comprise one or more of the following ingredients: about 89% of water, about 3% of propanediol, about 2% of glyceryl/polyglyceryl-3 caprylic/capric/lactic/lauric acid esters, about 2% of polyglyceryl-10 caprylate/caprate, about 2% of glyceryl monostearate, and about 2% of erucamide or derivatives thereof.

In another embodiment, a body lotion formulation according to the present disclosure comprises erucamide or derivatives thereof. The body lotion formulation may further comprise one or more of the following ingredients in the weight percentage ranges identified herein: water, glycerin, sodium phytate, sodium stearoyl glutamate, polyglycerin-3 (and) polyglyceryl-3 lactate/laurate, glyceryl stearate, caprylic/capric triglyceride, isoamyl laurate, squalane, Simmondsia chinensis (jojoba) seed oil, trioctyldodecyl citrate, oleth-2, butyrospermum parkii (shea) butter, hydroxethyl acrylate/sodium acryloyldimethyl taurate copolymer (and) isohexadecane (and) polysorbate 60, deionized water, butylene glycol, sodium hyaluronate, and docanoyl and octanoyl esters. Specifically, the body lotion formulation may comprise one or more of the following ingredients: about 66.20% of water, about 3% of glycerin, about 0.10% of sodium phytate, about 0.50% of sodium stearoyl glutamate, about 2% of polyglycerin-3 (and) polyglyceryl-3 lactate/laurate, about 3% of glyceryl stearate, about 5% of caprylic/capric triglyceride, about 4% of isoamyl laurate, about 2% of squalane, about 1% of Simmondsia chinensis (jojoba) seed oil, about 1% of trioctyldodecyl citrate, about 1.20% of oleth-2, about 3% of butyrospermum parkii (shea) butter, about 3% of hydroxethyl acrylate/sodium acryloyldimethyl taurate copolymer (and) isohexadecane (and) polysorbate 60, about 1% of deionized water, butylene glycol, sodium hyaluronate, about 2% of docanoyl and octanoyl esters, and about 2% of erucamide or derivatives thereof.

In another embodiment, a first deodorant stick formulation according to the present disclosure comprises erucamide or derivatives thereof. The first deodorant stick formulation may further comprise one or more of the following ingredients in the weight percentage ranges identified herein: magnesium hydroxide, Cocos nucifera (coconut) oil, arrowroot powder, butyrospermum parkii (shea) butter, caprylic/capric triglyceride, sodium bicarbonate, and Helianthus annuus (sunflower) seed wax. Specifically, the first deodorant stick formulation may comprise one or more of the following ingredients: about 24% magnesium hydroxide, about 18% Cocos nucifera (coconut) oil, about 20% arrowroot powder, about 10% butyrospermum parkii (shea) butter, about 2% erucamide, about 10% caprylic/capric triglyceride, about 6% sodium bicarbonate, and about 10% Helianthus annuus (sunflower) seed wax.

In another embodiment, a second deodorant stick formulation according to the present disclosure comprises erucamide or derivatives thereof. The second deodorant stick formulation may have a different hardness than the first deodorant stick formulation. The second deodorant stick formulation may further comprise one or more of the following ingredients in the weight percentage ranges identified herein: magnesium hydroxide, Cocos nucifera (coconut) oil, arrowroot powder, butyrospermum parkii (shea) butter, caprylic/capric triglyceride, sodium bicarbonate, and Helianthus annuus (sunflower) seed wax. Specifically, the second deodorant stick formulation may comprise one or more of the following ingredients: about 14% magnesium hydroxide, about 34% Cocos nucifera (coconut) oil, about 18% arrowroot powder, about 5% butyrospermum parkii (shea) butter, about 5% erucamide, about 10% caprylic/capric triglyceride, about 6% sodium bicarbonate, and about 8% Helianthus annuus (sunflower) seed wax.

In another embodiment, a second 2-in-1 conditioning shampoo formulation according to the present disclosure comprises erucamide or derivatives thereof. The second 2-in-1 conditioning shampoo formulation may further comprise one or more of the following ingredients in the weight percentage ranges identified herein: deionized water, cetyl hydroxyethylcellulose, sodium C14-16 olefin sulfonate, sodium lauroyl methyl isethionate, polyglycerin-3 (and) polyglyceryl-3 lactate/laurate, sodium lauroyl lactylate, cocamidopropyl betaine, glyceryl/polyglyceryl-3 caprylic/capric/lactic/lauric acid esters, polyglyceryl-10 caprylate/caprate, citric acid, and fragrance. Specifically, the second 2-in-1 conditioning shampoo formulation may comprise one or more of the following ingredients: about 45% deionized water, about 0.8% cetyl hydroxyethylcellulose, about 15% sodium C14-16 olefin sulfonate, about 10% sodium lauroyl methyl isethionate, about 8% polyglycerin-3 (and) polyglyceryl-3 lactate/laurate, about 2% sodium lauroyl lactylate, about 8% cocamidopropyl betaine, about 2% glyceryl/polyglyceryl-3 caprylic/capric/lactic/lauric acid esters, about 4% polyglyceryl-10 caprylate/caprate, about 4% erucamide, about 0.2% citric acid, and about 1% fragrance.

In another embodiment, a press powder eye shadow formulation according to the present disclosure comprises erucamide or derivatives thereof. The press powder eye shadow formulation may further comprise one or more of the following ingredients in the weight percentage ranges identified herein: caprylic/capric triglyceride, polyglyceryl-4 oleate, tocopheryl acetate, mica, mica (and) silica, mica (and) boron nitride, magnesium stearate, and titanium dioxide. Specifically, the press powder eye shadow formulation may comprise one or more of the following ingredients: about 4.25% caprylic/capric triglyceride, about 1.75% polyglyceryl-4 oleate, about 1% tocopheryl acetate, about 33% mica, about 25% mica (and) silica, about 15% mica (and) boron nitride, about 10% magnesium stearate, about 5% erucamide, and about 5% titanium dioxide.

It should be understood and appreciated that any personal care products, including, but not limited to, hair conditioning lotions, hair conditioners, conditioning shampoos, 2-in-1 conditioning shampoos, hair color treating shampoos and/or conditioners, hair color treating 2-in-1 conditioning shampoos, quat-free conditioners, non-foaming shave lotions and shave creams, foaming shave lotions and shave creams, brushless shave creams and lotions, moisturizing lotions and creams, barrier repair lotions and creams, conditioning hand lotions and creams, cleansing wipes, hair detangling sprays and/or leave-in conditioners, deodorants, deodorant sticks, antiperspirants, antiperspirant sticks, and pigmented products such as make-up, color cosmetics, powders, lipsticks, eye shadows, blush, foundation, etc. may have a formulation comprising erucamide or derivatives thereof as well as any other ingredient disclosed herein (and ingredients not specifically disclosed), in various concentrations and/or combinations thereof.

### EXAMPLES

Example 1: A Quat-Free Hair Conditioning Formulation of the Present Disclosure was prepared comprising the ingredients set forth in Table 1 below.

TABLE 1

| Ingredient | % (w/w) |
|---|---|
| Deionized water | 87.00 |
| Glycerin | 3.00 |
| Hydroxypropylmethylcellulose | 1.00 |
| Glyceryl monostearate | 2.00 |
| Erucamide | 2.00 |
| Cetyl alcohol | 3.00 |
| Glyceryl/polyglyceryl-3 | 2.00 |
| caprylic/capric/lactic/lauric acid esters | |
| pH | 5.25 |
| Viscosity (cps) | 14841 |

The formulation of Example 1 was prepared in a vessel by the following procedure:
1) adding the deionized water to the vessel,
2) adding the glycerin and dissolving it therein,
3) dispersing the hydroxypropylmethylcellulose into the mixture,
4) heating the mixture to 80° C.,
5) adding the glyceryl monostearate to the mixture,
6) adding the cetyl alcohol to the mixture,
7) adding the erucamide to the mixture;
8) adding the glyceryl/polyglyceryl-3 caprylic/capric/lactic/lauric acid esters to the mixture;
9) mixing the mixture until it is uniform;
10) cooling the mixture to below 30° C. and qs to 100%; and
11) mixing the cooled mixture until it is uniform.

The resulting formulation of Example 1 exhibited a pH of 5.25 and a viscosity of 14841 (cps), using a Brookfield LVDVII+with speed set to 6 rpm for 1 minute with the Helipath setting on.

The formulation of Example 1 was applied to the hair of a test subject after the hair was shampooed, and rinsed, as typically performed. The hair exhibited much improved wet and dry characteristics and was soft, shiny and more flowing and manageable, with less product build-up when compared to other hair conditioner products.

Example 2: A 2-in-1 conditioning shampoo of the present disclosure was prepared comprising the ingredients set forth in Table 2 below.

TABLE 2

| Ingredient | % (w/w) |
|---|---|
| Deionized water | 40.80 |
| Cetyl Hydroxyethylcellulose | 1.00 |
| Sodium C14-16 olefin sulfonate | 20.00 |
| Sodium lauroyl methyl isethionate | 10.00 |
| Sodium lauroyl sarcosinate | 10.00 |
| Cocamidopropyl betaine | 8.00 |
| Glyceryl/polyglyceryl-3 | 2.00 |
| caprylic/capric/lactic/lauric acid esters | |
| Polyglyceryl-10 caprylate/caprate | 4.00 |
| Erucamide | 4.00 |
| Citric Acid | 0.20 |

Example 3: A shave cream of the present disclosure was prepared comprising the ingredients set forth in Table 3 below.

TABLE 3

| Ingredient | % (w/w) |
|---|---|
| Deionized water | 79.00 |
| Stearic acid | 10.00 |
| Glyceryl/polyglyceryl-3 | 2.00 |
| caprylic/capric/lactic/lauric acid esters | |
| Polyglyceryl-10 caprylate/caprate | 2.00 |
| Erucamide | 2.00 |
| Glyceryl monostearate | 2.00 |
| Cetyl alcohol | 1.00 |
| Potassium hydroxide | 2.00 |

Example 4: A brushless shave cream of the present disclosure was prepared comprising the ingredients set forth in Table 4 below.

TABLE 4

| Ingredient | % (w/w) |
|---|---|
| Deionized water | 91.00 |
| Glyceryl/polyglyceryl-3 | 2.00 |
| caprylic/capric/lactic/lauric acid esters | |
| Polyglyceryl-10 caprylate/caprate | 2.00 |
| Erucamide | 2.00 |
| Glyceryl monostearate | 2.00 |
| Cetyl alcohol | 1.00 |

Example 5: A cleansing wipe of the present disclosure was prepared comprising the ingredients set forth in Table 5 below.

TABLE 5

| Ingredient | % (w/w) |
|---|---|
| Deionized water | 90.00 |
| Glyceryl/polyglyceryl-3 | 2.00 |
| caprylic/capric/lactic/lauric acid esters | |
| Polyglyceryl-10 caprylate/caprate | 4.00 |
| Erucamide | 2.00 |
| Sodium stearoyl lactylate | 2.00 |

Example 6: A hair detangling spray (e.g., leave-in conditioner) of the present disclosure was prepared comprising the ingredients set forth in Table 6 below.

TABLE 6

| Ingredient | % (w/w) |
|---|---|
| Deionized water | 89.00 |
| Propanediol | 3.00 |
| Glyceryl/polyglyceryl-3 | 2.00 |
| caprylic/capric/lactic/lauric acid esters | |
| Polyglyceryl-10 caprylate/caprate | 2.00 |
| Erucamide | 2.00 |
| Glyceryl monostearate | 2.00 |

Example 7: A body lotion of the present disclosure was prepared comprising the ingredients set forth in Table 7 below.

TABLE 7

| Ingredient | % (w/w) |
|---|---|
| Deionized water | 66.20 |
| Glycerin | 3.00 |
| Sodium phytate | 0.10 |
| Sodium stearoyl glutamate | 0.50 |
| Polyglycerin-3 (and) polyglyceryl-3 | 2.00 |
| lactate/laurate | |
| Glyceryl stearate | 3.00 |
| Caprylic/capric triglyceride | 5.00 |
| Isoamyl laurate | 4.00 |
| Squalane | 2.00 |
| Simmondsia chinensis (jojoba) seed oil | 1.00 |
| Trioctyldodecyl citrate | 1.00 |
| Oleth-2 | 1.20 |
| Butyrospermum parkii (shea) butter | 3.00 |
| Erucamide | 2.00 |
| Hydroxyethyl acrylate/sodium | 3.00 |
| acryloyldimethyl taurate copolymer | |
| (and) isohexadecane (and) polysorbate | |
| 60 | |
| Deionized water, butylene glycol, | 1.00 |
| sodium hyaluronate | |
| Docanoyl and octanoyl esters | 2.00 |

Example 8: The first deodorant stick formulation of the present disclosure was prepared comprising the ingredients set forth in Table 8 below.

TABLE 8

| Ingredient | % (w/w) |
|---|---|
| Magnesium Hydroxide | 24.00 |
| Cocos Nucifera (Coconut) Oil | 18.00 |
| Arrowroot Powder | 20.00 |
| Butyrospermum Parkii (Shea) Butter | 10.00 |
| Erucamide | 2.00 |
| Caprylic/Capric Triglyceride | 10.00 |
| Sodium Bicarbonate | 6.00 |
| Helianthus Annuus (Sunflower) Seed Wax | 10.00 |

Example 9: The second deodorant stick formulation of the present disclosure was prepared comprising the ingredients set forth in Table 9 below.

TABLE 9

| Ingredient | % (w/w) |
|---|---|
| Magnesium Hydroxide | 14.00 |
| Cocos Nucifera (Coconut) Oil | 34.00 |
| Arrowroot Powder | 18.00 |
| Butyrospermum Parkii (Shea) Butter | 5.00 |
| Erucamide | 5.00 |
| Caprylic/Capric Triglyceride | 10.00 |
| Sodium Bicarbonate | 6.00 |
| Helianthus Annuus (Sunflower) Seed Wax | 8.00 |

The formulation of Examples 8 and 9 were prepared in a vessel by the following procedure:
1) adding Cocos Nucifera (Coconut) Oil, Caprylic/Capric Triglyceride, Helianthus Annuus (Sunflower) Seed Wax, Butyrospermum Parkii (Shea) Butter to the vessel,
2) heating the mixture to 85° C.,
3) adding erucamide and mixing the mixture until it is uniform,
4) with moderate mixing, adding Magnesium Hydroxide, Arrow root Powder, and Sodium Bicarbonate to the mixture,
5) mixing the mixture until it is uniform,
6) cooling the mixture to 70° C.,
7) removing the mixture from heat and continuing cooling,
8) keep mixing while pouring the mixture into a container at 60° C.

Example 10: the second 2-in-1 conditioning shampoo formulation of the present disclosure was prepared comprising the ingredients set forth in Table 10 below.

TABLE 10

| Ingredient | % (w/w) |
|---|---|
| Deionized Water | 45.00 |
| Cetyl Hydroxyethylcellulose | 0.80 |
| Sodium C14-16 Olefin Sulfonate | 15.00 |
| Sodium Lauroyl Methyl Isethionate | 10.00 |
| Polyglycerin-3 (and) Polyglyceryl-3 | 8.00 |
| Lactate/Laurate | 2.00 |
| Sodium Lauroyl Lactylate | |
| Cocamidopropyl Betaine | 8.00 |
| Glyceryl/Polyglyceryl-3 | 2.00 |
| Caprylic/Capric/Lactic/Lauric Acid | |
| Esters | |
| Polyglyceryl-10 Caprylate/Caprate | 4.00 |
| Erucamide | 4.00 |
| Citric Acid | 0.20 |
| Fragrance | 1.00 |
| pH | 5.15 |
| Viscosity (cps) | 23330 |

In one embodiment, the formulation of Example 10 was prepared in a vessel by the following procedure:
1) adding the polyglyceryl-10 caprylate/caprate and the polyglycerin-3 (and) polyglyceryl-3 lactate/laurate to the vessel to combine them,
2) dispersing the cetyl hydroxyethylcellulose into the mixture,
3) heating the mixture to 80° C.,
4) adding the erucamide to the mixture,
5) mixing the mixture until melted and uniform,
6) adding the sodium c14-16 olefin sulfonate, the sodium lauroyl methyl isethionate, and the cocamidopropyl betaine to the mixture,
7) mixing and maintaining the mixture at 80° C. until the mixture is completely uniform,
8) adding the deionized water to the mixture,
9) maintaining the mixture at 50° C. until the mixture is completely uniform,
10) adding the citric acid and the glyceryl/polyglyceryl-3 caprylic/capric/lactic/lauric acid esters to the mixture,
11) cooling the mixture to 40° C. while mixing,
12) adding the fragrance to the mixture and qs to 100%,
13) mixing the fragrance to room temperature.

In another embodiment, the formulation of Example 10 was prepared in a vessel by the following procedure:
1) adding the deionized water to the vessel
2) dispersing the cetyl hydroxyethylcellulose into the mixture,
3) begin heating the mixture to 80° C.,
4) adding the sodium c14-16 olefin sulfonate, sodium lauroyl methyl isethionate, polyglycerin-3 (and) polyglyceryl-3 lactate/laurate, and the polyglyceryl-10 caprylate/caprate to the mixture,
5) at 80° C., adding the erucamide to the mixture,
6) maintaining the mixture at 80-85° C. and mixing until the erucamide is fully emulsified,
7) begin cooling the mixture to 40° C.,
8) adding the citric acid and the fragrance to the mixture and qs to 100%,
9) mixing the fragrance to room temperature.

In one embodiment, the formulation of Example 10 may have color retention properties.

Example 11: the press powder eye shadow formulation of the present disclosure was prepared comprising the ingredients set forth in Table 11 below.

TABLE 11

| Ingredient | % (w/w) |
|---|---|
| Phase A | |
| Caprylic/Capric Triglyceride | 4.25 |
| Polyglyceryl-4 Oleate | 1.75 |
| Tocopheryl Acetate | 1.00 |
| Phase B | |
| Mica | 33.00 |
| Mica (and) silica | 25.00 |
| Mica (and) boron nitride | 15.00 |
| Magnesium Stearate | 10.00 |
| Erucamide | 5.00 |
| Titanium Dioxide | 5.00 |

In one embodiment, the formulation of Example 11 was prepared in a vessel by the following procedure:
1) adding the titanium oxide to a mortar,
2) adding the Phase B ingredients (omitting Mica) to the mortar and mixing well after each addition,
3) adding the Phase A ingredients to the mortar and blending well until all ingredients are mixed and the color appears uniform,
4) adding the mica and blending well,
5) filling the eye shadow into eye shadow packaging and pressing the packaging.

The above detailed descriptions of embodiments of the present disclosure are not intended to be exhaustive or to limit the disclosure to the precise form disclosed herein. Although specific embodiments of, and examples for, the disclosure herein are described above for illustrative purposes, various equivalent modifications are possible within the scope of the disclosure, as those skilled in the relevant art will recognize. For example, while formulations comprise certain ingredients in certain amounts, alternative embodiments may incorporate additional ingredients not necessarily disclosed herein, be comprised of fewer ingredients, or have varying amounts of each ingredient without departing from the present disclosure. Further, while steps are presented in a given order, alternative embodiments may perform steps in a different order. The various embodiments described herein may also be combined to provide further embodiments applicable to a wide range of compositions.

Further, while advantages associated with certain embodiments of the technology have been described in the context of those embodiments, other embodiments may also exhibit such advantages, and not all embodiments need necessarily exhibit such advantages to fall with the scope of this disclosure. Accordingly, this disclosure and associated inventions can encompass other embodiments not expressly shown or described herein.

What is claimed is:

1. A personal care product comprising a formulation having erucamide or a derivative thereof.

2. The personal care product according to claim 1, wherein the formulation comprises about 0.0001% to about 20% by weight of erucamide or a derivative thereof.

3. The personal care product according to claim 1, wherein the formulation further comprises water.

4. The personal care product according to claim 3, wherein the formulation comprises about 0.0001% to about 98% by weight of water.

5. The personal care product according to claim 1, wherein the formulation further comprises glycerin.

6. The personal care product according to claim 5, wherein the formulation comprises about 0.0001% to about 20% by weight of glycerin.

7. The personal care product according to claim 1, wherein the formulation further comprises hydroxypropylmethylcellulose.

8. The personal care product according to claim 7, wherein the formulation comprises about 0.0001% to about 20% by weight of hydroxypropylmethylcellulose.

9. The personal care product according to claim 1, wherein the formulation further comprises glyceryl monostearate.

10. The personal care product according to claim 9, wherein the formulation comprises about 0.0001% to about 20% by weight of glyceryl monostearate.

11. The personal care product according to claim 1, wherein the formulation further comprises cetyl alcohol.

12. The personal care product according to claim 11, wherein the formulation comprises about 0.0001% to about 20% by weight of cetyl alcohol.

13. The personal care product according to claim 1 wherein the formulation further comprises glyceryl/polyglyceryl-3 caprylic/capric/lactic/lauric acid esters.

14. The personal care product according to claim 13, wherein the formulation comprises about 0.0001% to about 20% by weight of glyceryl/polyglyceryl-3 caprylic/capric/lactic/lauric acid esters.

15. The personal care product according to claim 1, wherein the personal care product is selected from the group consisting of: a hair conditioning lotion, a hair conditioner, a conditioning shampoo, a 2-in-1 conditioning shampoo, a hair color treating shampoo, a hair color treating conditioner, a hair color treating 2-in-1 conditioning shampoo, a quat-free conditioner, a non-foaming shave lotion, a non-foaming shave cream, a foaming shave lotion, a foaming shave cream, a brushless shave cream, a brushless shave lotion, a moisturizing lotion, a moisturizing cream, a barrier repair lotion, a barrier repair cream, a conditioning hand lotion, a conditioning hand cream, a cleansing wipe, a hair detangling spray, a leave-in conditioner, a deodorant, a deodorant stick, an antiperspirant, an antiperspirant stick, make-up, a press powder eye shadow, a color cosmetic, a powder, a lipstick, an eye shadow, a blush, and a foundation.

16. A method of formulating a personal care product comprising:
1) adding deionized water to a vessel;
2) adding glycerin to the vessel and dissolving the glycerin therein, forming a mixture;
3) dispersing Hydroxypropylmethylcellulose into the mixture;
4) heating the mixture to between 70-90° C.;
5) adding glyceryl monostearate to the mixture,
6) adding cetyl alcohol to the mixture,
7) adding erucamide or a derivative thereof to the mixture;
8) adding glyceryl/polyglyceryl-3 caprylic/capric/lactic/lauric acid esters to the mixture;
9) mixing the mixture until uniform;
10) cooling the mixture to 20-40° C. and qs to 100%; and
11) mixing the mixture until uniform,
wherein the water comprises about 87% of the formulation, the glycerin comprises about 3% of the formulation, the hydroxypropylmethylcellulose comprises about 1% of the formulation, the glyceryl monostearate comprises about 2% of the formulation, the erucamide or derivative thereof comprises about 2% of the formulation, the cetyl alcohol comprises about 3% of the formulation, and the glyceryl/polyglyceryl-3 caprylic/capric/lactic/lauric acid esters comprises about 2% of the formulation.

17. A personal care product comprising a formulation, the formulation comprising:
water having a concentration between 0.0001%-98%;
glycerin having a concentration between 0.0001%-20%;
hydroxypropylmethylcellulose having a concentration between 0.0001%-20%;
glyceryl monostearate having a concentration between 0.0001%-20%;
erucamide or a derivative thereof having a concentration between 1%-3%;
cetyl alcohol having a concentration of about 0.0001%-20%; and
glyceryl/polyglyceryl-3 caprylic/capric/lactic/lauric acid esters having a concentration of about 0.0001%-20%.
